(19) United States
(12) Patent Application Publication
(10) Pub. No.: US 2023/0285550 A1
(43) Pub. Date: Sep. 14, 2023

(54) COMPOSITIONS AND METHODS FOR VACCINE DELIVERY

(71) Applicant: Dianomi Therapeutics, Inc., Madison, WI (US)

(72) Inventors: Martin Ostrowski, Madison, WI (US); Anna B. Clements, Middleton, ID (US); Jae Sung Lee, Middleton, WI (US)

(21) Appl. No.: 18/106,357

(22) Filed: Feb. 6, 2023

Related U.S. Application Data
(63) Continuation of application No. PCT/US2021/044162, filed on Aug. 2, 2021.
(60) Provisional application No. 63/062,098, filed on Aug. 6, 2020.

(51) Int. Cl. A61K 39/39

(52) U.S. Cl. CPC A61K 39/39; A61K 2039/575

(57) ABSTRACT

In an aspect, provided herein is a method for vaccinating a subject in need thereof. In another aspect, the present disclosure provides a method for enhancing the cell-mediated immunity response of a viral antigen. In another aspect, the present disclosure provides a method for stabilizing a biological macromolecule. In another aspect, the present disclosure provides a vaccine composition. In another aspect, the present disclosure provides a stabilized composition comprising lyophilized mineral coated microparticles (MCM) bound to a biological macromolecule.

### CROSS-REFERENCE

This application is a continuation of International Application No. PCT/US2021/044162, filed Aug. 2, 2021, which claims priority to U.S. Provisional Patent Application No. 63/062,098, filed Aug. 6, 2020, both of which are incorporated herein by reference in their entirety for all purposes.

### BACKGROUND

Adequate vaccine performance can be especially difficult for subunit vaccines which are a fragment of a pathogen, typically a surface protein (e.g., antigen), that is used to trigger an immune response and stimulate acquired immunity against the pathogen from which it is derived. In some cases, the antigen is expressed from a nucleic acid (e.g., RNA vaccine) that is administered to the subject. In these cases, the subject's body expresses the antigen protein from the nucleic acid vaccine. However, duration of antigen expression and low antigen concentrations can present challenges for the use of certain vaccines, especially RNA vaccines and/or subunit vaccines.

### SUMMARY

Recognized herein is a need for vaccines with improved stability (e.g., for long-term storage and delivery globally without the need of refrigeration or freezing). This need is particularly pressing for instances of global pandemic caused by novel pathogens, for containment or eradication of pathogens, or in preparation for seasonal infections.

Also recognized herein is a need for improved vaccine effectiveness including improved bioavailability, improved immunogenicity, improved infectivity, improved humoral and/or cell-mediated immune response, or improved long-term memory immunity. This improved effectiveness can be achieved from a single dose or administration of a vaccine without the need for subsequent or booster doses.

As described herein, the need for improved vaccine stability, humoral and immunogenic response, including for subunit vaccines such as RNA vaccines is addressed by using a mineral-coated microparticle (MCM). MCMs can also improve the humoral or immunogenic response for other kinds of vaccines such as inactivated or attenuated viruses, conjugate vaccines, and the like. An MCM is a biomimetic, tailorable, mineral coated microparticle. MCMs can bind (e.g., adsorb), stabilize, and release proteins, peptides and nucleic acid molecules. As such, MCMs can be used as an excipient material to improve subunit vaccine formulations by prolonged delivery of antigen peptides and proteins for an extend time period (e.g., during germinal center initiation). In some cases, use of MCMs as described herein can improve humoral immunity and antibody responses. Furthermore, the addition of MCMs to mRNA vaccine formulations can allow for sequestration and subsequent sustained presence of the translated antigen peptide/proteins. The MCM can additionally function as an adjuvant to improve the immune response to the translated antigen.

MCMs can be constructed of generally regarded as safe (GRAS) materials. They can also be added to a vaccine formulation and optionally lyophilized to create a vaccine product that is stable at room temperature, can be stockpiled, and can be distributed without need for refrigeration. The lyophilized composition can be reconstituted and used at the point of administration.

In an aspect, provided herein is a method for vaccinating a subject in need thereof. The method can include (a) providing a formulation comprising a subunit vaccine molecule; (b) admixing the formulation with a mineral coated microparticle (MCM) to provide a vaccine, which MCM adsorbs the subunit vaccine molecule and has a diameter suitable for performing as an adjuvant when administered to a subject in need of vaccination; and (c) administering the vaccine to a subject in need of vaccination.

In some embodiments, the vaccine is injected into the subject.

In some embodiments, the vaccine is injected into a muscle of the subject.

In some embodiments, the vaccine has an improved bioavailability when compared with the formulation without an MCM.

In some embodiments, the vaccine has an improved immunogenicity when compared with the formulation without an MCM.

In some embodiments, the vaccine has an improved infectivity when compared with the formulation without an MCM.

In some embodiments, the vaccine antigen has a longer half-life when compared with the formulation without the MCM.

In some embodiments, the vaccine has an improved humoral response when compared with the formulation without an MCM.

In some embodiments, the vaccine elicits an improved long-term memory immunity when compared with the formulation without an MCM.

In some embodiments, a single dose of the vaccine is administered to the subject, wherein administration of the formulation to the subject can require a plurality of administrations to be effective.

In some embodiments, the subunit vaccine molecule is a protein or peptide.

In some embodiments, the subunit vaccine molecule is a nucleic acid.

In some embodiments, the MCM has a diameter less than about 100 um.

In some embodiments, the MCM has a core comprising calcium phosphate.

In some embodiments, the subunit vaccine molecule adsorbs upon and/or within a surface of the MCM.

In some embodiments, the vaccine is delivered as a plurality of subunit vaccine molecules and a plurality of MCM.

In some embodiments, a portion of the subunit vaccine molecules adsorb to the plurality of MCM.

In some embodiments, the dose of vaccine comprises of both adsorbed subunit vaccine molecules to MCM and unadsorbed subunit vaccine molecules.

In certain embodiments of the first aspect, the inorganic precipitate comprises calcium and phosphate ions in a molar ratio of from about 10:1 to about 1:10.

In another aspect, the present disclosure provides a method for enhancing the cell-mediated immunity response of a viral antigen. The method can include (a) providing a formulation comprising a viral antigen molecule; (b) admixing the formulation with mineral coated microparticles (MCM), wherein the antigen adsorbs to the MCM; and (c) administering to vertebrate subject a therapeutically effective amount of the formulation, wherein the formulation enhances the cell-mediated immune response against a target intracellular pathogen.

In some embodiments, the viral antigen molecule is a protein or peptide.

In some embodiments, the viral antigen molecule is expressed from a nucleic acid subsequent to administering the formulation to the vertebrate subject.

In some embodiments, the MCM has a diameter less than about 100 um.

In some embodiments, the MCM has a core comprising calcium phosphate.

In some embodiments, the viral antigen molecule adsorbs upon and/or within a surface of the MCM.

In some embodiments, the formulation enhances the cell-mediated immune response against a target intracellular pathogen when compared to administration of the viral antigen molecule without adsorption to the MCM.

In another aspect, the present disclosure provides a method for stabilizing a biological macromolecule. The method can comprise (a) creating a mixture comprising biological macromolecules and a mineral coated microparticles (MCM), wherein the biological macromolecule adsorbs to the MCM; (b) optionally removing biological macromolecules that are not adsorbed to the MCM from the mixture; and (c) lyophilizing the mixture to create a stabilized formulation.

In some embodiments, the stabilized formulation further comprises a pharmaceutically acceptable excipient material.

In some embodiments, the non-adsorbed biological macromolecules are removed by washing the MCM.

In some embodiments, the method further comprises reconstituting the stabilized formulation.

In some embodiments, the stabilized formulation is reconstituted in a solution suitable for administration to a subject in need thereof, optionally near the time and place of administration.

In some embodiments, the method further comprises administering an effective amount of the reconstituted formulation to a subject in need thereof.

In some embodiments, the reconstituted formulation is administered by intra-muscular injection of the subject.

In some embodiments, the biological macromolecule is a protein or peptide.

In some embodiments, the biological macromolecule is a nucleic acid.

In some embodiments, the MCM has a diameter less than about 100 um.

In some embodiments, the MCM has a core comprising calcium phosphate.

In some embodiments, the mixture comprises modified simulated body fluid (mSBF) comprising at least about 5 mM calcium ions and at least about 2 mM phosphate ions.

In some embodiments, the mixture has a pH of at least about 6.8.

In some embodiments, the adsorption is electrostatic.

In some embodiments, the biological macromolecule adsorbs upon and/or within a surface of the MCM.

In another aspect, the present disclosure provides a vaccine composition. The composition can comprise (a) subunit vaccine molecules; and (b) mineral coated microparticles (MCM), which MCM binds with the subunit vaccine molecules and has a diameter suitable for performing as an adjuvant when administered to a subject in need of vaccination.

In some embodiments, the vaccine composition further comprises an adjuvant.

In some embodiments, the subunit vaccine molecules comprise a polypeptide.

In some embodiments, the polypeptide has a sequence that is substantially similar to a viral protein or portion thereof.

In some embodiments, the polypeptide is attached to a polysaccharide.

In some embodiments, the subunit vaccine molecules comprise a nucleic acid.

In some embodiments, the nucleic acid encodes a polypeptide that has a sequence that is substantially similar to a viral protein or portion thereof.

In some embodiments, the nucleic acid is modified to increase its stability in a vaccine formulation.

In some embodiments, the nucleic acid is modified to enhance its expression when administered to a subject in need of vaccination.

In some embodiments, the composition further comprises a nucleic acid complexing agent.

In some embodiments, the complexing agent is selected from the group consisting of a polymer, a lipid and an adjuvant.

In some embodiments, the adjuvant is selected from the group consisting of an aluminum, an emulsion and a salt.

In another aspect, the present disclosure provides a stabilized composition comprising lyophilized mineral coated microparticles (MCM) bound to a biological macromolecule.

In some embodiments, the composition comprises less than about 5 weight-% water.

In some embodiments, the MCM has a diameter less than about 100 um.

In some embodiments, the composition is suitable for reconstitution in an aqueous medium for administration to a subject in need thereof.

In some embodiments, the composition remains at least 90% active after six months at room temperature.

In some embodiments, at least about 90% of the biological macromolecule is active upon reconstitution of the composition.

In some embodiments, the biological macromolecule is a protein or peptide.

In some embodiments, the biological macromolecule is a nucleic acid.

In some embodiments, the MCM has a diameter less than about 100 um.

In some embodiments, the MCM has a core comprising calcium phosphate.

In some embodiments, the biological macromolecule adsorbs upon and/or within a surface of the MCM.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a micrograph of a mineral coated microparticle (MCM);

FIG. 2 shows a phase diagram used to show the principal of lyophilization;

FIG. 3 shows a schematic drawing of a MCM used in combination with a mRNA vaccine;

FIG. 4A shows a conceptual graph of serum concentration of a subunit vaccine over time for bolus delivery;

FIG. 4B shows a conceptual graph of serum concentration of a subunit vaccine over time for delivery using the sustained release technology described herein;

FIG. 5A shows a differential scanning calorimetry plot;

FIG. 5B shows a differential scanning calorimetry plot;

FIG. 5C shows a differential scanning calorimetry plot; and

FIG. 5D shows a differential scanning calorimetry plot.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The compositions and methods described herein can be used to vaccinate a subject in need of vaccination. In some cases, the storage and stabilization of subunit vaccines is improved. Subunit vaccines can have stability concerns, limited shelf life, and require cold chain shipping and storage (2-8° C.). As described herein, mineral-coated microparticles (MCMs) can attenuate these issues by stabilizing peptides and proteins in harsh conditions, preserving both their structure and activity. MCMs can bind (adsorb) proteins within the nanostructured coating surface through electrostatic interactions between the proteins' charged/polar groups and the coating and have a tailorable loading capacity as high as 0.8 mg protein/1 mg MCMs. Unlike encapsulation technologies such as PLGA or PEG, manufacture with MCMs is simple and scalable, as proteins are loaded onto the surface of the MCM in aqueous solution instead of organic solvents, making formulation less complex and costly. Additionally, proteins loaded onto MCMs can be stabilized during storage, preventing protein aggregation and deactivation seen with other encapsulation technologies. In some cases, the MCM-adsorbed vaccine formulation can be lyophilized (freeze dried) for additional stabilization. In some cases, the MCM stabilize the protein during the lyophilization process and/or during storage after lyophilization.

The compositions and methods described herein can also be used to improve the immunogenicity of vaccines by sustained delivery of the antigen. MCMs can sustain the release of active proteins and peptides in vivo. In mRNA formulations, MCMs have an advantage of sequestering and then sustaining delivery of the encoded protein, which is useful where the duration of antigen expression and maintenance of antigen concentration are crucial. Ultimately, for subunit vaccines (including where those subunits are expressed by an mRNA vaccine), global manufacturing capabilities are faced with overwhelming international supply and demand concerns. The MCMs' ability to provide sustained delivery of vaccine antigens can allow for a more effective immunological vaccine response and, therefore, simultaneously reduce potential shortage concerns.

The addition of mineral coated microparticles as an excipient material to subunit and mRNA-based vaccines can improve the humoral immune and antibody response, e.g., as an adjuvant-type material. MCMs can be added at escalating concentrations to the vaccine formulation and administered to generate a therapeutically effective dose. The dosing route can be injected (e.g., intramuscular or subcutaneous), applied topically, or inhaled (e.g., nasal/aerosol delivery). Humoral and antibody responses can be examined and compared to a once administered vaccine control group and a multidose schedule vaccine control group to determine the effect. The MCM can sequester and deliver translated proteins in vivo following subcutaneous and intramuscular administration of mRNA-based formulations (e.g., in a human or animal model) and determine the ability of MCMs to function as an adjuvant material by examining the influx of antigen presenting cells to the site of administration.

### Definitions

Unless defined otherwise, all technical and scientific terms used herein have the same meanings as commonly understood by one of skill in the art to which the disclosure herein belongs. As used herein, the singular forms “a,” “an,” and “the,” are intended to include the plural forms as well, unless the context clearly indicates otherwise. The phrase “and/or,” as used herein in the specification and in the claims, should be understood to mean “either or both” of the elements so conjoined, e.g., elements that are conjunctively present in some cases and disjunctively present in other cases. Thus, as a nonlimiting example, a reference to “A and/or B”, when used in conjunction with open-ended language such as “comprising” can refer, in some embodiments, to A only (optionally including elements other than B); in some embodiments, to B only (optionally including elements other than A); in some embodiments, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in some embodiments, to at least one, optionally including more than one, B, with no A present (and optionally including elements other than A); in some embodiments, to at least one, optionally including more than one, A, and at least one, optionally including more than one, B (and optionally including other elements); etc. In certain embodiments, the term “about” or “approximately” as used herein means within an acceptable error range for the particular value as determined, which will depend in part on how the value is measured or determined, e.g., the limitations of the measurement system.

In certain embodiments, “about” can mean within 3 or more than 3 standard deviations, per the practice in the art. In certain embodiments, such as with respect to biological systems or processes, the term can mean within an order of magnitude, including within 5-fold, and within 2-fold of a value. In certain embodiments, when the term “about” or “approximately” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and subrange within that range. For example, “1-5 ng” or “from about 1 ng to about 5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “administering,” refers to the placement of the vaccine dose as disclosed herein into a subject by a method or route which results in at least partial delivery of the composition at an appropriate extracellular location of a target tissue. In certain embodiments, the vaccine dose adsorbed to the MCM component can be, for example, injected into a subject in need thereof by either intradermal, intra-muscular, subcutaneous, intra-articular, peri-articular or intravenous administration. In certain embodiments, the vaccine dose adsorbed to the MCM component administered parenterally, e.g., by intravenous, intra-arterial, intracardiac, intraspinal, intraosseous, intra-articular, intra-synovial, subcutaneous, intradermal, intra-tendinous, intraligamentous or intramuscular administration. In certain embodiments, the bioactive compound captured within the inorganic precipitate is administered by implantation, infiltration or infusion.

The therapeutically effective amount can vary depending upon the intended application or the subject and disease condition being treated, e.g., the weight and age of the subject, the severity of the disease condition, the manner of administration and the like, which can readily be determined such as by a board-certified physician.

As used herein, the terms “treat,” “treatment,” “treating” refer to therapeutic treatments, wherein the object is to reverse, alleviate, ameliorate, inhibit, slow down or stop the progression or severity of a disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disorder is reduced or halted. That is, “treatment” includes not just the improvement of symptoms or markers, but also a cessation of, or at least slowing of, progress or worsening of symptoms compared to what would be expected in the absence of treatment. Beneficial clinical results include, but are not limited to, alleviation of one or more symptom(s), diminishment of extent of disease, stabilized (e.g., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “tissue” or “target tissue” refers to an aggregation of morphologically similar cells and associated intercellular matter, e.g., extracellular matrix, acting together to perform one or more specific functions in the body. In some embodiments, tissues fall into one of four basic types: muscle, nerve, epidermal, and connective. In some embodiments, a tissue is substantially solid, e.g., cells within the tissue are strongly associated with one another to form a multicellular solid tissue. In some embodiments, a tissue is substantially non-solid, e.g., cells within the tissue are loosely associated with one another, or not at all physically associated with one another, but may be found in the same space, bodily fluid, etc.

As used herein, a ceramic can be neither metallic nor organic material that can be crystalline, glassy or both crystalline and glassy. In certain embodiments, ceramics can be hard and chemically non-reactive and can be formed or densified with heat.

As used herein, the term “extracellular” means being situated or taking place outside a cell or cells.

A “subject” refers to a vertebrate, such as a mammal (e.g., a non-human mammal), such as a primate or a human. Mammals include, but are not limited to, primates, humans, farm animals, rodents, sport animals, and pets.

As used herein, a “subunit vaccine” is a fragment of a pathogen, such as a surface protein, that is used to trigger an immune response and stimulate acquired immunity against the pathogen from which it is derived.

As used herein, “transfection” is the process of deliberately introducing nucleic acids into eukaryotic cells. Transfection of animal cells can involve opening transient pores or holes in the cell membrane to allow the uptake of material. Transfection can be carried out in vitro using calcium phosphate (e.g., hydroxyapatite, tricalcium phosphate), by electroporation, by cell squeezing or by mixing a cationic lipid with the nucleic acids to produce liposomes that fuse with the cell membrane and deposit their cargo inside. Transfection in vivo can be more difficult than in vitro and can be improved by the use of MCM's as described in U.S. Patent Pub. No. 2016/0017368 A1, which is incorporated herein by reference in its entirety for all purposes.

As used herein, “bioavailability” is a fraction (%) of an administered drug that reaches the systemic circulation. By definition, when a medication is administered intravenously, its bioavailability is 100%. However, when a medication is administered via routes other than intravenous, its bioavailability can be lower than that of intravenous. In some cases, bioavailability equals the ratio of comparing the area under the plasma drug concentration curve versus time (AUC) for the extravascular formulation to the AUC for the intravascular formulation. In some cases, to ensure that the drug taker who has poor absorption is dosed appropriately, the bottom value of the deviation range is employed to represent real bioavailability to calculate drug dose for the drug taker to achieve systemic drug concentrations similar to the intravenous formulation. To dose without the prerequisite of drug taker's absorption state, the bottom value of the deviation range can be used in order to ensure the anticipated efficacy will be met unless the drug is associated with narrow therapeutic window. Bioavailability can be measured over any suitable period of time.

As used herein, the term “formulation”, generically indicates the beneficial agents and mineral coated microparticles are formulated, mixed, added, dissolved, suspended, solubilized, formulated into a solution, carried and/or the like in or by the fluid, gas, or solid in a physical-chemical form acceptable for patient administration.

“Effective amount” or “therapeutically effective amount” means a dosage sufficient to alleviate one or more symptoms of the condition being treated, or to otherwise provide a pharmacological and/or physiologic effect, as may be determined by an objective measure or a patient derived subjective measure. In certain embodiments, an “effective amount” refers to the optimal amount of a vaccine dose adsorbed to the MCM needed to elicit a clinically significant improvement in the symptoms and/or pathological state associated with a disease state, infection, or disorder to be treated. In certain embodiments the disease state, infection, or disorder to be treated is a viral pathogen. In certain embodiments, the vaccine dose adsorbed to the MCM is administered as a treatment. In certain embodiments, the vaccine dose adsorbed to the MCM is administered prophylactically as a preventative measure. As used herein, an “effective amount”, a “therapeutically effective amount”, a “prophylactically effective amount” and a “diagnostically effective amount” is the amount of the unbound active agent and the active agent adsorbed to the mineral coated microparticle needed to elicit a biological response following administration.

As used herein, “a subject in need thereof” (also used interchangeably herein with “a patient in need thereof”) refers to a subject susceptible to or at risk of a specified disease, disorder, or condition. The methods disclosed herein can be used with a subset of subjects who are susceptible to or at elevated risk of infection by a condition for which the vaccine is provided. Because some of the method embodiments of the present disclosure are directed to specific subsets or subclasses of identified subjects (that is, the subset or subclass of subjects “in need” of assistance in addressing or vaccinating against one or more specific conditions noted herein), not all subjects will fall within the subset or subclass of subjects as described herein for certain diseases, disorders or conditions.

As used herein, “immunogenicity” is the ability of a foreign substance, such as an antigen, to provoke an immune response in the body of a human or other animal. In other words, immunogenicity is the ability to induce a humoral and/or cell-mediated immune responses. The immune system is divided into a more primitive innate immune system, and acquired or adaptive immune system of vertebrates, each of which contains humoral and cellular components.

As used herein, “humoral immunity” is the aspect of immunity that is mediated by macromolecules found in extracellular fluids such as secreted antibodies, complement proteins, and certain antimicrobial peptides. Humoral immunity is so named because it involves substances found in the humors, or body fluids. It contrasts with cell-mediated immunity. Its aspects involving antibodies are often called antibody-mediated immunity. Humoral immunity refers to antibody production and the accessory processes that accompany it, including: Th2 activation and cytokine production, germinal center formation and isotype switching, affinity maturation and memory cell generation. It also refers to the effector functions of antibodies, which include pathogen and toxin neutralization, classical complement activation, and opsonin promotion of phagocytosis and pathogen elimination.

As used herein, “cell-mediated immunity” is an immune response that does not involve antibodies. Rather, cell-mediated immunity is the activation of phagocytes, antigen-specific cytotoxic T-lymphocytes, and the release of various cytokines in response to antigen. CD4 cells or helper T cells provide protection against different pathogens. Naive T cells, which are immature T cells that have yet to encounter an antigen, are converted into activated effector T cells after encountering antigen-presenting cells (APCs). These APCs, such as macrophages, dendritic cells, and B cells in some circumstances, load antigenic peptides onto the MHC of the cell, in turn presenting the peptide to receptors on T cells. The most important of these APCs are highly specialized dendritic cells; conceivably operating solely to ingest and present antigens. Activated Effector T cells can be placed into three functioning classes, detecting peptide antigens originating from various types of pathogen: The first class being 1) Cytotoxic T cells, which kill infected target cells by apoptosis without using cytokines, 2) TH1 cells, which primarily function to activate macrophages, and 3) TH2 cells, which primarily function to stimulate B cells into producing antibodies. Cellular immunity protects the body through: (a) T-cell mediated immunity or T-cell immunity: activating antigen-specific cytotoxic T cells that are able to induce apoptosis in body cells displaying epitopes of foreign antigen on their surface, such as virus-infected cells, cells with intracellular bacteria, and cancer cells displaying tumor antigens; (b) macrophage and natural killer cell action: enabling the destruction of pathogens via recognition and secretion of cytotoxic granules (for natural killer cells) and phagocytosis (for macrophages); and (c) stimulating cells to secrete a variety of cytokines that influence the function of other cells involved in adaptive immune responses and innate immune responses.

### Subunit Vaccines

A subunit vaccine presents an antigen to the immune system without introducing whole or disabled viral particles. One method of production can involve isolation of a specific protein from a virus and administering this by itself. A potential weakness of this technique is that isolated proteins can be denatured and then become associated with antibodies different from target antibodies. A second potential method of making a subunit vaccine can involve putting an antigen's gene from the targeted virus or bacterium into another virus (virus vector), yeast (yeast vector), as in the case of the hepatitis B vaccine or attenuated bacterium (bacterial vector) to make a recombinant virus or bacteria to serve as the main component of a recombinant vaccine (called a recombinant subunit vaccine). The recombinant vector that is genomically modified will express the antigen. The antigen (one or more subunits of protein) can be extracted from the vector. Just like the highly successful subunit vaccines, the recombinant-vector-produced antigen can be of less risk to the patient. This is the type of vaccine currently in use for hepatitis B, and it is experimentally popular, being used to try to develop new vaccines for difficult-to-vaccinate-against viruses such as SARS-Cov-2, ebolavirus and HIV.

Another type of subunit vaccine is the Vi capsular polysaccharide vaccine (ViCPS). This can contain the signature polysaccharide linked to the Vi capsular antigen. It is also called a conjugate vaccine, in which a polysaccharide antigen has been covalently attached to a carrier protein for T-cell-dependent antigen processing (utilizing MEW II).

An RNA vaccine or mRNA vaccine is another type of vaccine for providing acquired immunity. Just like other vaccines, RNA vaccines can induce the production of antibodies which will bind to potential pathogens. The RNA sequence codes for antigens, proteins that are identical or resembling those of the pathogen. Upon the delivery of the vaccine into the body, this sequence is translated by the host cells to produce the encoded antigens, which then stimulate the body's adaptive immune system to produce antibodies against the pathogen. In some cases, these translated peptides or proteins are subunits of vaccine proteins. RNA vaccines offer multiple potential advantages over DNA vaccines in terms of production, administration, and safety, and can be therapeutic in humans. RNA vaccines are also thought to have the potential to be used for cancer in addition to infectious diseases. In some cases, RNA vaccines are delivered through an RNA containing vector, such as lipid nanoparticles.

In some cases, the subunit vaccine (including mRNA vaccines) include an adjuvant. As used herein, an “adjuvant” is a pharmacological or immunological agent that improves the immune response of a vaccine. Adjuvants may be added to a vaccine to boost the immune response to produce more antibodies and longer-lasting immunity, thus minimizing the dose of antigen needed. Adjuvants may also be used to enhance the efficacy of a vaccine by helping to modify the immune response to particular types of immune system cells: for example, by activating T cells instead of antibody-secreting B cells depending on the purpose of the vaccine. There are different classes of adjuvants that can affect the immune response in different ways, including adjuvants include aluminum hydroxide and paraffin oil. Without limitation, adjuvants suitable for use in the materials and methods described herein include: analgesic adjuvants; inorganic compounds (alum, aluminium hydroxide, aluminium phosphate, calcium phosphate hydroxide); mineral oil (paraffin oil); bacterial products (killed bacteria Bordetella pertussis, Mycobacterium bovis, toxoids); non-bacterial organics (squalene); detergents (Quil A); plant saponins (quillaja, soybean, polygala senega); cytokines (IL-1, IL-2, IL-12); Freund's complete or incomplete adjuvant; or food-based oil (adjuvant 65, which is a product based on peanut oil).

### Mineral Coated Microparticles

As described herein, the MCM can stabilize macromolecules and/or act as an adjuvant. The MCM can include compounds within or on its surface that are adjuvants. The diameter of the MCM can also be tailored to elicit an immune response. In some embodiments, the diameter of the MCM is about 1 micrometer (um), about 3 um, about 5 um, about 10 um, about 30 um, about 50 um, about 80 um, about 100 um, about 120 um, about 150 μm, about 200 um, about 300 um, or about 500 um. In some embodiments, the diameter of the MCM is at least about 1 micrometer (um), at least about 3 um, at least about 5 um, at least about 10 um, at least about 30 um, at least about 50 um, at least about 80 um, at least about 100 um, at least about 120 um, at least about 150 um, at least about 200 um, at least about 300 um, or at least about 500 um. In some embodiments, the diameter of the MCM is at most about 1 micrometer (um), at most about 3 um, at most about 5 um, at most about 10 um, at most about 30 um, at most about 50 um, at most about 80 um, at most about 100 um, at most about 120 um, at most about 150 um, at most about 200 um, at most about 300 um, or at most about 500 um. The diameter of the MCM can be tailored by using a larger or smaller core material or by the conditions of deposition of the mineral coating on the core material, which conditions can include time of reaction or concentration of components in the simulated body fluid solution (described below).

The MCM can also be an excipient. As used herein, an “excipient” is a substance formulated alongside the active ingredient of a medication, included for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life. The selection of appropriate excipients also depends upon the route of administration and the dosage form, as well as the active ingredient and other factors. The MCM can be a stabilizer to increase the half-life of a therapeutic, either alone or in combination with other excipients.

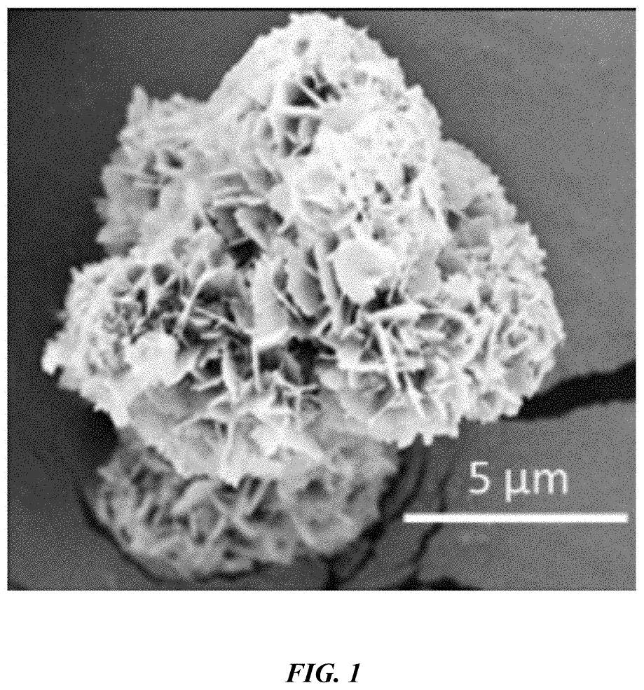

FIG. 1 shows a micrograph of an example MCM having a nano-structured calcium phosphate mineral coating. These can provide a platform for sustained delivery of biological macromolecules such as subunit vaccines. The mineral coated microparticles offer an injectable and systemic or localized delivery system that can lower the dose and off-target side-effects when compared to bolus injection of vaccines, such as with subunit vaccines such as mRNA vaccine having short half-lives or having reduced activity. The formulations and methods disclosed herein advantageously allow for both immediate effect of the vaccine that is delivered in unbound form, as well as sustained effect of the vaccine by adsorbing the vaccine (and/or mRNA expression products) to mineral coated microparticles that provide sustained delivery of the antigen as the mineral coating degrades and releases the antigen. Other active agents can be incorporated into the mineral coated microparticles or within a carrier solution to improve the delivery of the vaccine subunit.

Mineral coated microparticles offer a delivery system that can sustainably release subunit vaccines while maintaining their activity. In some cases, these microparticles can remain localized when injected in vivo and offer a localized delivery system which can allow for lower therapeutic dosages when compared to systemic subcutaneous or intravenous delivery. Further, release of vaccine from mineral coated microparticles can be tailored by altering the coating composition. In addition, mineral coated microparticles have a high binding capacity for biological macromolecules which allows them to sustainably deliver a suitable dose of vaccine subunit with little delivery system material. This may widen the applicability of sustained delivery systems for subunit vaccines.

In some embodiments, the formulation includes a mineral coated microparticle, wherein the mineral coated microparticle comprises a core; a mineral coating on the core; and a vaccine subunit. In some embodiments, the core is a nucleation site for coating precipitation. In some embodiments, the vaccine subunit is adsorbed to the mineral coating. In some embodiments only the vaccine subunit is incorporated throughout the mineral coating. In some embodiments, there are layers of mineral coating on the core. In some embodiments, the vaccine subunit is adsorbed to multiple layers of mineral coating on the core. In some embodiments, multiple, different active agents are adsorbed to the mineral coating along with a vaccine subunit. In some embodiments, multiple vaccine subunits are adsorbed to the mineral coating. The vaccine subunits can be different types of antigens.

In some embodiments, the formulation includes a mineral coated microparticle, wherein the mineral coated microparticle comprises a core, a first layer of mineral coating on the core, an active agent such as a vaccine subunit adsorbed onto the first layer of mineral coating, a second layer of mineral coating and a second active agent such as a vaccine subunit adsorbed to the second layer of mineral coating. In some embodiments, the active agent adsorbed onto the first layer of mineral coating is the same as the active agent adsorbed on the second layer of mineral coating. In some embodiments, the active agent adsorbed onto the first layer of mineral coating is different than the active agent adsorbed on the second layer of mineral coating. In some embodiments, more than one active agent is adsorbed on each layer of mineral coating (e.g., 2, 3, 4, 5, 6, 7, 8, 9, 10, or more active agents). In some embodiments, at least one of the active agents adsorbed on any of the layers of mineral coating is a vaccine subunit. In some embodiments, all of the active agents adsorbed on the layers of mineral coating are vaccine subunits.

There can be additional active agents in the fluid in which the MCMs are suspended (“carrier”) during their manufacture and/or upon administration. In some embodiments, the formulation includes a carrier, wherein the carrier is for a mineral coated microparticle, wherein the mineral coated microparticle comprises a core; a mineral coating on the core; and a vaccine subunit adsorbed to the mineral coating. In some embodiments, another active agent is adsorbed to the mineral coating along with the vaccine subunit. In some embodiments, the carrier is a liquid. In some embodiments, the carrier is a solution or a liquid. In some embodiments, the carrier is a gel. In some embodiments the carrier is a gas. In some embodiments, the carrier is a solid. In some embodiments, the carrier contains an active agent. In some embodiments, the active agent is a vaccine subunit. In some embodiments, the active agent in the carrier contains the same vaccine subunit adsorbed on or incorporated within the mineral coating. In some embodiments, the active agent in the carrier is a different vaccine subunit than the vaccine subunit adsorbed on or incorporated within the mineral coating. In some embodiments, the carrier contains more than one active agent. In some embodiments, the carrier contains multiple vaccine subunits. In some embodiments, the carrier contains a vaccine subunit and one or more active agents that are not vaccine subunits.

In some embodiments, the at least one of the active agents adsorbed to the mineral coating is the same as the active agent in the carrier. In some embodiments, the active agents adsorbed to the mineral coating are all different from the active agent in the carrier. In another aspect, at least two different active agents are adsorbed to the mineral coating. Contemplated embodiments further include 2, 3, 4, 5 or more different active agents adsorbed to the mineral coating. In some embodiments, the active agent incorporated within the mineral coating is the same as the active agent in the carrier. In some embodiments, the active agent incorporated within the mineral coating is different from the active agent in the carrier. In another aspect, at least two different active agents are incorporated within the mineral coating. Contemplated embodiments further include 2, 3, 4, 5 or more different active agents incorporated within the mineral coating. At least one of the active agents is a vaccine subunit. In another aspect, an active agent can be incorporated within the mineral coating in combination with an active agent adsorbed to the mineral coating. Formulations include 2, 3, 4, 5 or more different active agents in the carrier solution.

Suitable liquid carriers include water, saline, isotonic saline, phosphate buffered saline, Ringer's lactate, and the like. Suitable gel carriers include collagen, hydrogels, polymer gels, polyethylene glycol, and the like.

Formulations can also include other components such as surfactants, preservatives, and excipients. Surfactants can reduce or prevent surface-induced aggregation of the active agent and the mineral coated microparticles. Various surfactants can be employed, such as polyoxyethylene fatty acid esters and alcohols, and polyoxyethylene sorbitol fatty acid esters. Amounts can range from about 0.001 and about 4% by weight of the formulation. Pharmaceutically acceptable preservatives include, for example, phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) and mixtures thereof. The preservative can be present in concentrations ranging from about 0.1 mg/ml to about 20 mg/ml, including from about 0.1 mg/ml to about 10 mg/ml. A preservative can be used in pharmaceutical compositions such as, but not limited to those described in “Remington: The Science and Practice of Pharmacy, 19th edition, 1995,” which is incorporated herein by reference in its entirety for all purposes. Formulations can include suitable buffers such as sodium acetate, glycylglycine, HEPES (4-(2-hydroxyethyl)-1-piperazineethanesulfonic acid) and sodium phosphate. Excipients include components for tonicity adjustment, antioxidants, and stabilizers used in the preparation of pharmaceutical formulations. Other inactive ingredients include, for example, L-histidine, L-histidine monohydrochloride monohydrate, sorbitol, polysorbate 80, sodium citrate, sodium chloride, and EDTA disodium.

Any suitable material can be used as the core upon which the mineral coating is formed. Suitable core materials include those materials non-toxic to humans and animals. Suitable core materials also include those materials that degrade and/or dissolve in humans and animals. Suitable core materials include β-tricalcium phosphate (β-TCP), hydroxyapatite (HA), poly(lactic-co-glycolic acid) (PLGA), and combinations thereof. β-tricalcium phosphate cores are can be suitable as the β-tricalcium phosphate degrades rapidly after mineral coating dissolution. Both β-tricalcium phosphate and hydroxyapatite are can also be suitable cores because they dissolve into calcium and phosphate ions which are easily metabolized by the body. In other embodiments, the core material can be dissolved following mineral coating formation. In other embodiments, the core material is non-degradable.

The mineral coating can include calcium, phosphate, carbonate, and combinations thereof. To prepare a mineral coated microparticle a core material is incubated in a modified simulated body fluid. Simulated body fluid contains the same ion constituents at the same concentrations as human blood plasma. Modified simulated body fluid contains similar, but altered ion constituents as human blood plasma. In some embodiments, the modified simulated body fluid contains twice the concentration of calcium and phosphate as human blood plasma along with the other ionic components of human blood plasma at physiological concentrations. The modified simulated body fluid can include calcium and phosphate, which form the mineral coating on the surface of the core, which results in the mineral coated microparticle. Because the modified simulated body fluid contains a supersaturation of calcium and phosphate, a mineral coating precipitates from solution onto the core material to form the mineral coating. Different mineral coating morphologies can be achieved by varying the amounts and ratios of calcium, phosphate, and carbonate in the modified simulated body solution during coating precipitation. Other ions, or dopants, can also be added to the modified simulated body fluid during coating formation to change the coating composition and/or morphology. Different mineral coating morphologies include, for example, plate-like structure, spherulite-like structure. High carbonate concentration can result in a mineral coating having a plate-like structure. Low carbonate concentration can result in a mineral coating having a spherulite-like structure. The mineral coating morphology can also affect adsorption of the active agent. The mineral coating morphology can also affect the preservation of activity of the active agent release from the mineral coating.

Suitable core materials on which the mineral coating is formed include polymers, ceramics, metals, glass and combinations thereof in the form of particles. Suitable particles can be, for example, agarose beads, latex beads, magnetic beads, polymer beads, ceramic beads, metal beads (including magnetic metal beads), glass beads and combinations thereof. The microparticle can include ceramics (e.g., hydroxyapatite, beta-tricalcium phosphate (beta-TCP, β-TCP), magnetite, neodymium), plastics (e.g., polystyrene, poly-caprolactone), hydrogels (e.g., polyethylene glycol; poly(lactic-co-glycolic acid), and the like, and combinations thereof. Suitable core materials can be those that dissolve in vivo such as, for example, beta-tricalcium phosphate (beta-TCP, β-TCP).

Suitable microparticle sizes can range from about 1 μm to about 100 μm in diameter. Microparticle diameter can be measured by, for example, measurements taken from microscopic images (including light and electron microscopic images), filtration through a size-selection substrate, and the like.

The modified simulated body fluid (mSBF) for use in the methods of the present disclosure can include from about 5 mM to about 12.5 mM calcium ions, including from about 7 mM to about 10 mM calcium ions, and including about 8.75 mM calcium ions; from about 2 mM to about 12.5 mM phosphate ions, including from about 2.5 mM to about 7 mM phosphate ions, and including from about 3.5 mM to about 5 mM phosphate ions; and from about 4 mM to about 100 mM carbonate ions.

In some embodiments, the mSBF can further include about 145 mM sodium ions, from about 6 mM to about 9 mM potassium ions, about 1.5 mM magnesium ions, from about 150 mM to about 175 mM chloride ions, about 4 mM HCO₃⁻, and about 0.5 mM SO₄²⁻ ions.

The pH of the mSBF can range from about 4 to about 7.5, including from about 5.3 to about 6.8, including from about 5.7 to about 6.2, and including from about 5.8 to about 6.1.

Suitable mSBF can include, for example: about 145 mM sodium ions, about 6 mM to about 9 mM potassium ions, about 5 mM to about 12.5 mM calcium ions, about 1.5 mM magnesium ions, about 150 mM to about 175 mM chloride ions, about 4.2 mM HCO₃⁻, about 2 mM to about 5 mM HPO₄²⁻ ions, and about 0.5 mM SO₄²⁻ ions. The pH of the simulated body fluid may be from about 5.3 to about 7.5, including from about 6 to about 6.8.

In some embodiments, the mSBF may include, for example: about 145 mM sodium ions, about 6 mM to about 17 mM potassium ions, about 5 mM to about 12.5 mM calcium ions, about 1.5 mM magnesium ions, about 150 mM to about 175 mM chloride ions, about 4.2 mM to about 100 mM HCO₃⁻, about 2 mM to about 12.5 mM phosphate ions, and about 0.5 mM SO₄²⁻ ions. The pH of the simulated body fluid may be from about 5.3 to about 7.5, including from about 5.3 to about 6.8.

In some embodiments, the mSBF includes: about 145 mM sodium ions, about 6 mM to about 9 mM potassium ions, from about 5 mM to about 12.5 mM calcium ions, about 1.5 mM magnesium ions, about 60 mM to about 175 mM chloride ions, about 4.2 mM to about 100 mM HCO₃⁻, about 2 mM to about 5 phosphate ions, about 0.5 mM SO₄²⁻ ions, and a pH of from about 5.8 to about 6.8, including from about 6.2 to about 6.8.

In some embodiments, the mSBF includes: about 145 mM sodium ions, about 9 mM potassium ions, about 12.5 mM calcium ions, about 1.5 mM magnesium ions, about 172 mM chloride ions, about 4.2 mM HCO₃⁻, about 5 mM to about 12.5 mM phosphate ions, about 0.5 mM SO₄²⁻ ions, from about 4 mM to about 100 mM CO₃²⁻, and a pH of from about 5.3 to about 6.0.

In embodiments that include a layered mineral coating, a core can be incubated in a formulation of modified simulated body fluid. The layer of mineral coating forms on the core during the incubation period of minutes to days. After the initial layer of mineral coating is formed on the core, the mineral coated microparticle can be removed from the modified simulated body fluid and washed. To form a plurality of layers of mineral coating a mineral coated microparticle can be incubated in a second, third, fourth, etc. modified simulated body fluid until an appropriate number of layers of mineral coating is achieved. During each incubation period a new layer of mineral coating forms on the previous layer. These operations are repeated until the appropriate number of layers of mineral coating is achieved.

During mineral formation, active agents such as vaccine subunits can be included in the modified simulated body fluid to incorporate active agents within the layer of mineral coating during mineral formation. The active agent can be a vaccine subunit or can be a different active agent. Following formation of each layer of mineral, the mineral coated microparticle can then be incubated in a carrier comprising at least one active agent to adsorb the agent to the layer of mineral coating. After incorporating an active agent within a layer of mineral coating and/or adsorbing an active agent to a layer of mineral coating, another layer of mineral coating can be formed by incubating the microparticle in another formulation of modified simulated body fluid. In some cases, layers of mineral coating can incorporate an active agent in the mineral, layers can have an active agent adsorbed to the layer of mineral, the layer of mineral coating can be formed without incorporating an active agent or adsorbing an active agent, and combinations thereof. Mineral coated microparticles having different layers of mineral coating can be prepared by forming a layer of mineral using one formulation of modified simulated body fluid, then incubating the mineral coated microparticle in a different formulation of modified simulated body fluid. Thus, mineral coated microparticles can be prepared to have a plurality of layers of mineral coating wherein each layer is different. Embodiments are also contemplated that include two or more layers of mineral coating that are the same combined with one or more layers of mineral coating that are the different. One of the active agents can be a vaccine subunit such as an antigen or an mRNA that expresses an antigen when administered to a subject in need of vaccination.

Tailoring the composition of the mineral coating in the different layers advantageously allows for tailored release kinetics of the active agent or active agents from each layer of the mineral coating. In embodiments where one or more active agents is incorporated within the mineral coating, the active agent can be included in the mSBF. As mineral formation occurs, active agents become incorporated with the mineral coating. In other embodiments, magnetic material can be incorporated into mineral coatings. For example, superparamagnetic iron oxide linked to bovine serum albumin can be incorporated into mineral coatings. Linked proteins (e.g., bovine serum albumin) can adsorb onto the mineral coating to incorporate the magnetic material with the mineral coating. In some embodiments, the mineral coating further includes a dopant. Suitable dopants include halogen ions, for example, fluoride ions, chloride ions, bromide ions, and iodide ions. The dopant(s) can be added with the other components of the mSBF prior to incubating the substrate in the mSBF to form the mineral coating. The dopant ions can alter the dissolution kinetics of the mineral and can thus alter the release kinetics of vaccine subunit or other active agent from the mineral coating.

In some embodiments, halogen ions including fluoride ions can be used. Suitable fluoride ions can be provided by fluoride ion-containing agents such as water-soluble fluoride salts, including, for example, alkali and ammonium fluoride salts. Incorporation of fluoride alters the stability of the mineral coating. The fluoride ion-containing agent can be included in the mSBF to provide an amount of up to 100 mM fluoride ions, including from about 0.001 mM to 100 mM, including about 0.01 mM to about 50 mM, including from about 0.1 mM to about 15 mM, and including about 1 mM fluoride ions. Inclusion of one or more dopants in the mSBF can result in the formation of a halogen-doped mineral coating that can have significantly different morphologies and/or dissolution and release kinetics. The different morphology may be beneficial for preserving the activity of the active agent release from the mineral coating. The control of mineral coating dissolution can be beneficial when tailoring the coating to have sufficient release kinetics for the active agent to enhance efficacy. In some embodiments, magnetic materials, including magnetite, magnetite-doped plastics, and neodymium, are used for the microparticle core material. Including magnetic materials results in the formation of MCM for which location and/or movement/positioning of the MCM by application of a magnetic force is enabled. The alternate use of magnetic microparticle core materials can allow for spatial control of where the active agent and/or the vaccine subunit is delivered. The mineral coatings may be formed by incubating the substrate with the mSBF at a temperature of about 37° C. for a period of time ranging from about 3 days to about 10 days.

To adsorb the vaccine subunit to the mineral coated microparticle, the mineral coated microparticles can be contacted with a solution containing the vaccine subunit. This contact can form a vaccine subunit loaded mineral coated microparticle. Other active agent(s) can also be adsorbed to the mineral coating along with the vaccine subunit by including them in the solution with the vaccine subunit. Alternatively, the microparticles can be contacted with a second solution containing other active agent(s) after loading with the vaccine subunit. Addition of other active agents can make the delivery of vaccine subunit more efficient or effective. In some embodiments, only a vaccine subunit is incorporated, adsorbed, or loaded onto or into the mineral coating. As used herein, “active agent” refers to a biologically active molecule. As used herein, “vaccine subunit loaded mineral coated microparticle” refers to a mineral coated microparticle which has vaccine subunit adsorbed to the mineral coating and/or has vaccine subunit incorporated throughout the coating. The vaccine subunit and/or other active agent(s) can be contacted with the mineral coated microparticle using any suitable method. For example, a solution of the vaccine subunit and/or other active agent(s) can be pipetted, poured, or sprayed onto the mineral coated microparticle. Alternatively, the mineral coated microparticle can be dipped in a solution including vaccine subunit and/or other active agent(s) along with the vaccine subunit. Alternatively, the mineral coated microparticle can be bathed or incubated in a solution containing vaccine subunit and/or other active agent(s). The vaccine subunit, and/or other active agent(s) can adsorb to the mineral coating by an electrostatic interaction between the vaccine subunit or active agent and the mineral coating of the mineral coated microparticle. Suitable active agents include biological molecules. Suitable active agents include proteins, small molecules, hormones, steroids, NSAIDs, cytokines, therapeutic proteins, antibodies, receptor antagonists, or the like. Adsorption of the vaccine subunit, or other active agents along with the vaccine subunit, to the mineral coated microparticles can be tailored by changing the mineral constituents (e.g., high carbonate and low carbonate microspheres), by changing the amount of mineral coated microparticles incubated with the vaccine subunit, or other active agents along, by changing the concentration of vaccine subunit, or other active agents in the incubation solution, and combinations thereof.

Additional details regarding methods for producing the modified simulated body fluid (mSBF) and/or for forming or binding molecules to the MCM can be found in “Addition of Mineral-Coated microparticles to soluble interleukin-1 receptor antagonist injected subcutaneously improves and extends systematic interleukin-1 inhibition” A.E.B. Clements, et. al., Advanced Therapeutics, vol. 1, issue 7, 1800048, November 2018; “Single-dose mRNA therapy via biomaterial-mediated sequestration of overexpressed proteins”, Khalil et al., Sci. Adv. 2020; 6; or “Nanostructured mineral coatings stabilize proteins for therapeutic delivery”, X. Yu, et al., Adv. Mater. 2017 September, 29(33), each of which is incorporated herein by reference in its entirety for all purposes.

After completing the mineral coating preparation, the mineral coatings can be analyzed to determine the morphology and composition of the mineral coatings. The composition of the mineral coatings can be analyzed by energy dispersive X-ray spectroscopy, Fourier transform infrared spectrometry, X-ray diffractometry, and combinations thereof. Suitable X-ray diffractometry peaks can be, for example, at 26° and 31°, which correspond to the (0 0 2) plane, the (2 1 1) plane, the (1 1 2) plane, and the (2 0 2) plane for the hydroxyapatite mineral phase. Suitable X-ray diffractometry peaks can be, for example, at 26° and 31°, which correspond to the (0 0 2) plane, the (1 1 2) plane, and the (3 0 0) plane for carbonate-substituted hydroxyapatite. Other suitable X-ray diffractometry peaks can be, for example, at 16°, 24°, and 33°, which correspond to the octacalcium phosphate mineral phase. Suitable spectra obtained by Fourier transform infrared spectrometry analysis can be, for example, a peak at 450-600 cm⁻¹, which corresponds to O—P—O bending, and a peak at 900-1200 cm⁻¹, which corresponds to asymmetric P—O stretch of the PO₄³⁻ group of hydroxyapatite. Suitable spectra peaks obtained by Fourier transform infrared spectrometry analysis can be, for example, peaks at 876 cm⁻¹, 1427 cm⁻¹, and 1483 cm⁻¹, which correspond to the carbonate (CO₃²⁻) group. The peak for HPO₄²⁻ can be influenced by adjusting the calcium and phosphate ion concentrations of the mSBF used to prepare the mineral coating. For example, the HPO₄²⁻ peak can be increased by increasing the calcium and phosphate concentrations of the mSBF. Alternatively, the HPO₄²⁻ peak can be decreased by decreasing the calcium and phosphate concentrations of the mSBF. Another suitable peak obtained by Fourier transform infrared spectrometry analysis can be, for example, a peak obtained for the octacalcium phosphate mineral phase at 1075 cm⁻¹, which can be influenced by adjusting the calcium and phosphate ion concentrations in the simulated body fluid used to prepare the mineral coating. For example, the 1075 cm⁻¹ peak can be made more distinct by increasing the calcium and phosphate ion concentrations in the simulated body fluid used to prepare the mineral coating. Alternatively, the 1075 cm⁻¹ peak can be made less distinct by decreasing the calcium and phosphate ion concentrations in the simulated body fluid used to prepare the mineral coating.

Energy dispersive X-ray spectroscopy analysis can also be used to determine the calcium/phosphorus ratio of the mineral coating. For example, the calcium/phosphorus ratio can be increased by decreasing the calcium and phosphate ion concentrations in the mSBF. Alternatively, the calcium/phosphorus ratio may be decreased by increasing the calcium and phosphate ion concentrations in the mSBF. Analysis of the mineral coatings by energy dispersive X-ray spectroscopy allows for determining the level of carbonate (CO₃²⁻) substitution for PO₄³⁻ and incorporation of HPO₄²⁻ into the mineral coatings The mSBF can include calcium and phosphate ions in a ratio ranging from about 10:1 to about 0.2:1, including from about 2.5:1 to about 1:1.

Further, the morphology of the mineral coatings can be analyzed by scanning electron microscopy, for example. Scanning electron microscopy can be used to visualize the morphology of the resulting mineral coatings. The morphology of the resulting mineral coatings can be, for example, a spherulitic microstructure, plate-like microstructure, and/or a net-like microstructure. Suitable average diameters of the spherulites of a spherulitic microstructure can range, for example, from about 2 μm to about 42 μm. Suitable average diameters of the spherulites of a spherulitic microstructure can range, for example, from about 2 μm to about 4 μm. In some embodiments, average diameters of the spherulites of a spherulitic microstructure can range, for example, from about 2.5 μm to about 4.5 μm. In some embodiments, average diameters of the spherulites of a spherulitic microstructure can range, for example, from about 16 μm to about 42 μm.

Mineral coated microparticles can be stored for later use, washed and stored for later use, washed and immediately used for adsorption, or immediately used for adsorption without washing. Storage of mineral coated microparticles can include lyophilization.

### Lyophilization

Lyophilization, also called freeze drying or cryodesiccation, is a low temperature dehydration process that involves freezing the product, lowering pressure, then removing the ice by sublimation. This is in contrast to dehydration by methods that evaporate water using heat. Lyophilization can result in a high quality vaccine product by avoiding high temperatures that can damage the protein or nucleic acid components.

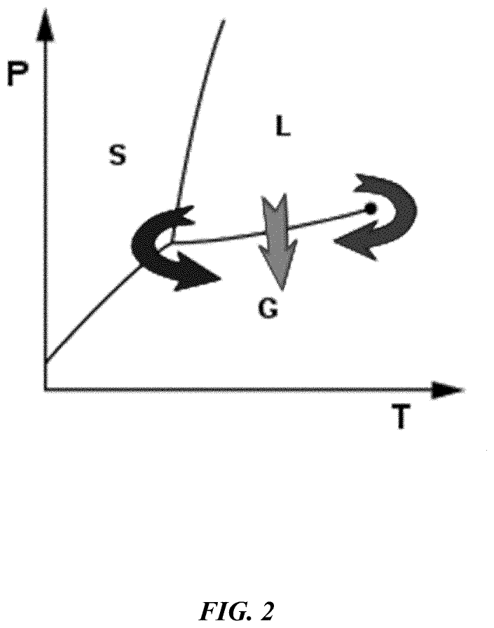

FIG. 2 shows an example phase diagram with variable pressure (P) and temperature (T). The phases include solid (S), liquid (L), and gas (G). The boundary between gas and liquid runs from the triple point to the critical point. Lyophilization brings the system around the triple point (left-most arrow) and avoids a direct liquid-gas transition of ordinary drying achieved by providing heat (middle arrow) or supercritical methods (right arrow).

In some cases, stages in a freeze drying process include: pretreatment, freezing, primary drying, and secondary drying. Pretreatment can include any method of treating the product prior to freezing. This may include concentrating the product, formulation revision (e.g., addition of components to increase stability, preserve appearance, and/or improve processing), decreasing a high-vapor-pressure solvent, or increasing the surface area.

During the freezing stage, the material can be cooled below its triple point, the lowest temperature at which the solid, liquid and gas phases of the material can coexist. This ensures that sublimation rather than melting can occur as follows. To facilitate faster and more efficient freeze drying, larger ice crystals can be used. The large ice crystals form a network within the product which promotes faster removal of water vapor during sublimation. To produce larger crystals, the product can be frozen slowly or can be cycled up and down in temperature in a process called annealing. The freezing phase can be the most critical in the whole freeze-drying process, as the freezing method can impact the speed of reconstitution, duration of freeze-drying cycle, product stability, and appropriate crystallization.

During the primary drying phase, the pressure can be lowered (to the range of a few millibars), and enough heat can be supplied to the material for the ice to sublime. The amount of heat to be supplied can be calculated using the sublimating molecule's latent heat of sublimation. In this initial drying phase, about 95% of the water in the material can be sublimated. This phase may be slow (e.g., several days), because, if too much heat is added, the material's structure can be altered. In this phase, pressure can be controlled through the application of partial vacuum. The vacuum can speed up the sublimation, making it useful as a deliberate drying process. Furthermore, a cold condenser chamber and/or condenser plates can provide a surface(s) for the water vapor to re-liquefy and solidify on.

The secondary drying phase can remove unfrozen water molecules, since the ice was removed in the primary drying phase. This part of the freeze-drying process is governed, at least in part, by the material's adsorption isotherms. In this phase, the temperature can be raised higher than in the primary drying phase, and can even be above 0° C. (32° F.), to break any physio-chemical interactions that have formed between the water molecules and the frozen material. The pressure is also lowered in this stage to encourage desorption (e.g., in the range of microbars, or fractions of a pascal). However, there are products that can benefit from increased pressure as well. After the freeze-drying process is complete, the vacuum can be broken with an inert gas, such as nitrogen, before the material is sealed. At the end of the operation, the residual water content in the product is extremely low, around 1% to 4%.

### Methods of Vaccination

In some cases, the vaccine subunits are adsorbed to the MCM, lyophilized, delivered, and reconstituted at the site of administration. In some embodiments, the vaccine formulation and MCMs can be constructed separately and then added together. MCMs have the ability to sequester secreted, translated gene products during therapeutic mRNA delivery, and can sustainably deliver intact and active proteins in multiple delivery scenarios both in vitro and in vivo. E.g., the immunological advantages of the MCM can be achieved without the long-term storage and stability advantages.

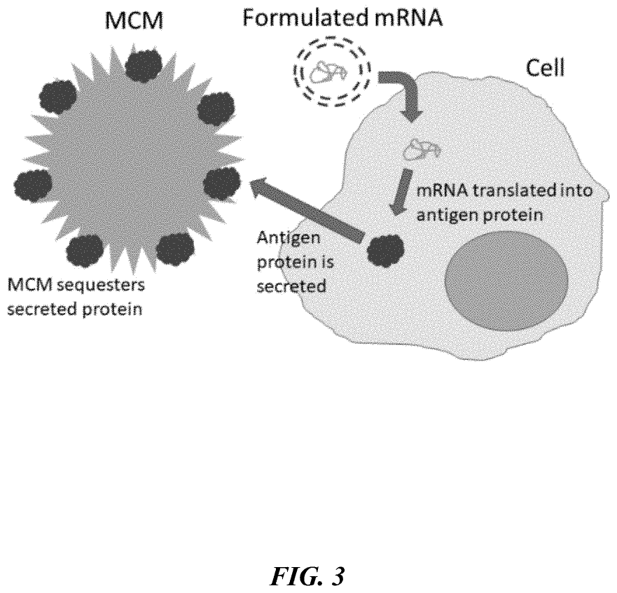

The lyophilized vaccine can be reconstituted in any suitable formulation, such as a formulation suitable for administration to a subject in need of vaccination. With reference to an example in FIG. 3, the mRNA transcript can be transfected into the cell. Once the mRNA transits to the cytosol, the intracellular translation machinery can produce a protein that undergoes post-translational modifications, resulting in a properly folded, fully functional protein. In some cases the mRNA transcript encodes for an mRNA vaccine that is intracellularly expressed as a subunit vaccine, and then excreted extracellularly by the cell. Following translation and secretion, the expressed subunit vaccine can be adsorbed to the MCM extracellularly. The MCM can be decomposed to its constituent ions over a period of time that extends the presentation of the antigen to the cell in order to develop a strong humoral and/or cell-mediated immune response.

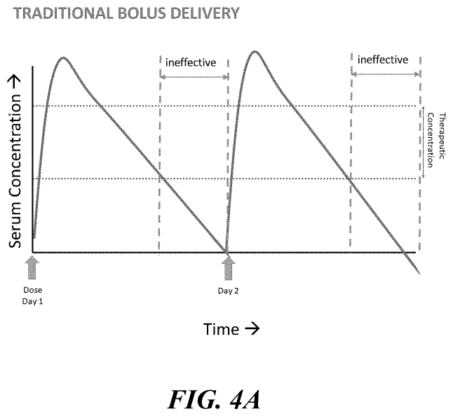

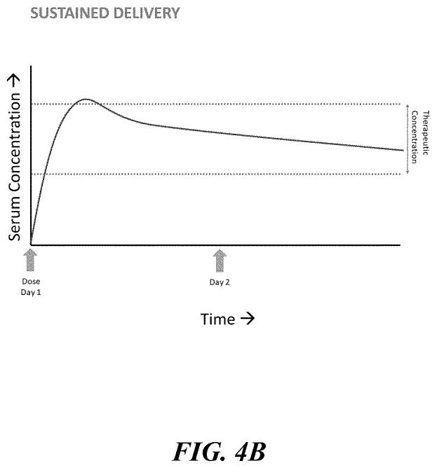

Binding of biomolecules to the MCMs can involve electrostatic interactions between the calcium and phosphate on the surface of the mineral coating and the polar or charged groups of the biomolecule. With reference to examples in FIG. 4A and FIG. 4B, addition of MCMs can improve the pharmacokinetics of already formulated biomolecules or biomolecules that are present in vivo. In some cases, a formulation without an MCM can have a short period of time at which the vaccine subunit is present at a therapeutic concentration (e.g., a concentration suitable for eliciting an immune response) and may require a plurality of doses (FIG. 4A). In contrast, as shown in FIG. 4B, vaccine subunits used in combination with MCMs can maintain the therapeutic concentration within an effective range for a longer period of time, optionally avoiding the need for a subsequent administration of the vaccine. As such, the MCMs are an attractive platform to control vaccine kinetics and can serve two unique and critical roles in subunit and/or mRNA-based vaccine sustainability.

First, MCMs can serve as an excipient material that, when added to the vaccine formulation, binds, stabilizes, and releases formulated antigens (e.g. peptides, proteins) or nucleic acid-based vaccines in a controlled and sustained manner after injection. The mineral surface serves as a platform for binding and stabilizing vaccines to the coating and maintaining conformational structure. This technique can be useful for mRNA-based vaccines where unformulated or naked RNA is easily degraded in vivo by ubiquitous RNAses. In some cases, the mRNA-based vaccine transcript is first complexed with a complexing agent prior to formulating with MCMs. In some embodiments, the complexing agent is a polymer, a lipid or an adjuvant that acts by binding and condensing mRNA through electrostatic interactions, forming mRNA complexes that can be internalized by the cells.

Second, MCMs can serve as a sequestering material for translated antigen peptide/proteins. MCMs initially used to deliver the mRNA-based vaccine sequesters the resulting translated peptide/protein thereby allowing for prolonged and sustained antigen presentation. The MCMs used to sequester antigens may additionally function as an adjuvant to improve the immune response to the mRNA-translated antigen. In some cases, the MCMs may serve to stimulate an independent inflammatory response, which can bolster the immunostimulatory effect of the target subunit protein antigen and/or mRNA-translated antigen therapeutic. In some cases, the MCMs elicit a transient macrophage response.

In some cases, the MCMs are loaded with an immunostimulatory molecule along with the subunit vaccine. Examples of immunostimulatory molecules include granulocyte-macrophage colony-stimulating factor (GM-CSF), macrophage colony-stimulating factor (M-CSF), or chemotactic agents for macrophages and dendritic cells. The stimulatory molecule can be bound and released by the MCM or included in the formulation.

Overall, MCMs can stabilize and/or sequester subunit or mRNA-based vaccines to substantially extend the duration of antigen activity such that only one single dose is therapeutic. The resulting sustained delivery of antigens during germinal center initiation significantly improves humoral and antibody response. This translates to a significant reduction in vaccine required for successful patient immunization, which is especially critical when vaccines are in high demand and/or short supply. MCMs are also relatively inexpensive to produce. GMP or Pharma grade materials to produce 1 kilogram of MCMs cost ˜$4,000, or an estimated $0.02 per 5 mg human dose.

Subunit vaccines can be used to stimulate humoral immunity but, without an adjuvant, can fail to induce cellular immunity, which can be required to eradicate the intracellular pathogen reservoir of many chronic diseases. Vaccines that are mRNA-based elicit a potent humoral and cellular immunity, but delivery of unprotected mRNA to the cell is prone to catalytic hydrolysis by ribonucleases. Moreover, mRNA has a short cytoplasmic half-life which limits the duration of protein production to hours which often necessitates repeated dosing. The use of MCMs in vaccine formulations can address these shortcomings of subunit vaccines.

The vaccine subunits and/or other active agent(s) adsorbed to the mineral coating of the mineral coated microparticle are released as the mineral coating degrades. Mineral degradation can be controlled such that the mineral coating can degrade rapidly or slowly. Mineral coating dissolution rates can be controlled by altering the mineral coating composition. For example, mineral coatings that possess higher carbonate substitution degrade more rapidly. Mineral coatings that possess lower carbonate substitution degrade more slowly. Incorporation of dopants, such as fluoride ions, may also alter dissolution kinetics. Alterations in mineral coating composition can be achieved by altering ion concentrations in the modified simulated body fluid during coating formation. Modified simulated body fluid with higher concentrations of carbonate, 100 mM carbonate for example, results in coatings which degrade more rapidly than coatings formed in modified simulated body fluid with physiological carbonate concentrations (4.2 mM carbonate).

Formulations for parenteral administration (e.g. by injection, for example bolus injection or continuous infusion) can be presented in unit dose form in ampoules, pre-filled syringes, small volume infusion or in multi-dose containers with and without an added preservative. The formulations can take such forms as suspensions, solutions, or emulsions in oily or aqueous vehicles, and may contain formulation agents such as suspending, stabilizing and/or dispersing agents. Alternatively, the mineral coated microparticles with active agent may be in powder form, obtained for example, by lyophilization from solution, for constitution with a suitable vehicle, e.g. sterile, pyrogen-free water, before use.

Vaccine subunits can be sustainably delivered with formulations which include mineral coated microparticles and vaccine subunits as vaccine subunits are released in a continuous manner as the coating dissolves. Other active agents can also be sustainably delivered along with the vaccine subunit when adsorbed to or incorporated in the mineral coating. The mineral coated microparticles can be delivered in a carrier solution containing an active agent to improve sustained delivery of the vaccine subunit.

Suitable methods for administration of formulations of the present disclosure are by parenteral (e.g., intramuscular, subcutaneous, intraperitoneal, or local injection into a tissue) administration routes. Local injection of the formulation into a tissue can be used to locally delivery vaccine subunits to a site where it is needed while decreasing systemic exposure to the vaccine subunits which may have unwanted side effects. In some embodiments, the formulation is administered through local injection into the synovium. In some embodiments, the formulation in injected intra-articularly to deliver steroid to the synovial fluid and/or the synovial lining. In some embodiments, the formulation is injected into an organ. Oral administration can also be used as a route of administration for the formulation containing mineral coated microparticles and a vaccine subunit. Oral administration can be utilized for sustained delivery of vaccine subunits in tissue of the digestive track, including the esophagus, the stomach, the small and large intestines, and the colon. Oral administration of the formulation containing mineral coated microparticles and a vaccine subunit can also be used for systemic administration of vaccine subunits. Inhaled administration can also be for delivery of the formulation of mineral coated microparticles and vaccine subunits. Inhaled administration may be used to locally deliver vaccine subunit to the lung or systemically delivery vaccine subunits. Administration routes and the formulations administered ordinarily include effective amounts of product in combination with acceptable diluents, carriers and/or adjuvants. Standard diluents such as human serum albumin are contemplated for pharmaceutical compositions of the invention, as are standard carriers such as saline.

Sustained delivery of the active agent, including the vaccine subunit, can be determined to obtain active agent release values that mimic established therapeutic levels of the active agent. The mass of mineral coated microparticles (with the vaccine subunit included) required to deliver a an appropriate concentration of the vaccine subunit over a period of time can be calculated beforehand. For example, a single bolus injection of the vaccine subunit that provides a therapeutic and/or immune effect can be delivered in a sustained manner over a period of time by obtaining the vaccine subunit release values from the mineral coated microparticles. Then the mass of mineral coated microparticles needed to deliver the vaccine subunit to provide the therapeutic or vaccine effect of a period of time can be calculated. The sustained delivery platform offers the benefit of continuous therapeutic or vaccination levels of the vaccine subunits without the requirement for multiple injections.

For nucleic acid formulations, the macromolecule can be encapsulated in a carrier (e.g., lipid nano-particle). In some cases, the formulation further comprises a transfection reagent. In such cases, the nucleic acid might not be directly bound to the MCM. In some instances, the carrier or transfection reagent is bound to the MCM.

In some embodiments, the nucleic acid is associated with a complexing agent. The complexing agent can be selected from the group consisting of a polymer, a lipid and an adjuvant. In some cases, the complexing agent interacts with and/or is bound to the MCM.

Effective dosages can vary substantially depending upon the vaccine subunits and other active agents. Because of the rapid and sustained delivery of the active agents contained in the formulations of the present disclosure, suitable dosages can be less than effective dosages of active agents delivered via bolus injections. As described herein, mineral coated microparticles can be prepared to deliver an effective amount of the vaccine subunit over the course of several days. Thus, administration of formulations of the instant application provide a bolus administration of unbound active agent that has a rapid effect and the sustained release of the active agent(s), including at least one vaccine subunits, during degradation of the mineral coating of the mineral coated microparticle has a sustained release of the steroid to maintain the effect over the course of time.

Formulations of the present disclosure can be administered to subjects in need thereof. As used herein, “a subject” (also interchangeably referred to as “an individual” and “a patient”) refers to animals including humans and non-human animals. Accordingly, the compositions, devices and methods disclosed herein can be used for human and veterinarian applications, including human and veterinarian medical applications. Suitable subjects include warm-blooded mammalian hosts, including humans, companion animals (e.g., dogs, cats), cows, horses, mice, rats, rabbits, primates, and pigs, and a human patient.

### EXAMPLES

### Example 1: Differential Scanning Calorimetry Analysis

Differential Scanning calorimetry (DSC) was performed on MCMs alone which shows that there are no phase transitions (besides freezing and melting of water). This analysis can inform the choice of parameters for lyophilization procedures for an MCM as described herein.

TABLE 1
Analysis Conditions

| | TEMPERATURE | TEMPERATURE | |
|---|---|---|---|
| | RANGE | CHANGE RATE | ANALYSIS |
| SAMPLE TYPE | (° C.) | (° C./min) | ENVIRONMENT |
| Coated Microparticles | -70° C. to 70° C. | 10 | Nitrogen |
| Core Material | | | |

TABLE 2
Data Summary

| | PHASE TRANSITION | | |
|---|---|---|---|
| | ONSET | PEAK | ENTHALPY OF |
| | TEMPERATURE | TEMPERATURE | TRANSITION |
| SAMPLE ID | (° C.) | (° C.) | (J/g) |

Coated Microparticles/ FN1000-1 mSBF 4.2 mM, Lot: 12/21/19-Wet

| | | | |
|---|---|---|---|
| Heating Cycle 1 | 1.2 | 23.0 | 356 |
| Cooling Cycle 1 | -28.3 | -31.6 | -244.8 |
| Heating Cycle 1 | 1.5 | 23.1 | 360.3 |

Core Material / Lot: 6/3/20-Wet

| | | | |
|---|---|---|---|
| Heating Cycle 1 | 1.2 | 21.2 | 356 |
| Cooling Cycle 1 | -22.0 | -27.1 | -260.9 |
| Heating Cycle 1 | 1.2 | 21.0 | 357 |

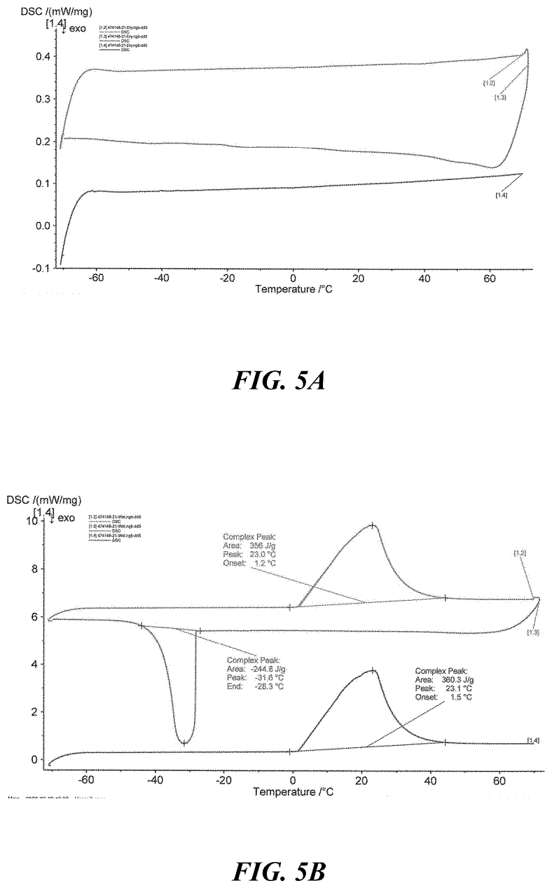

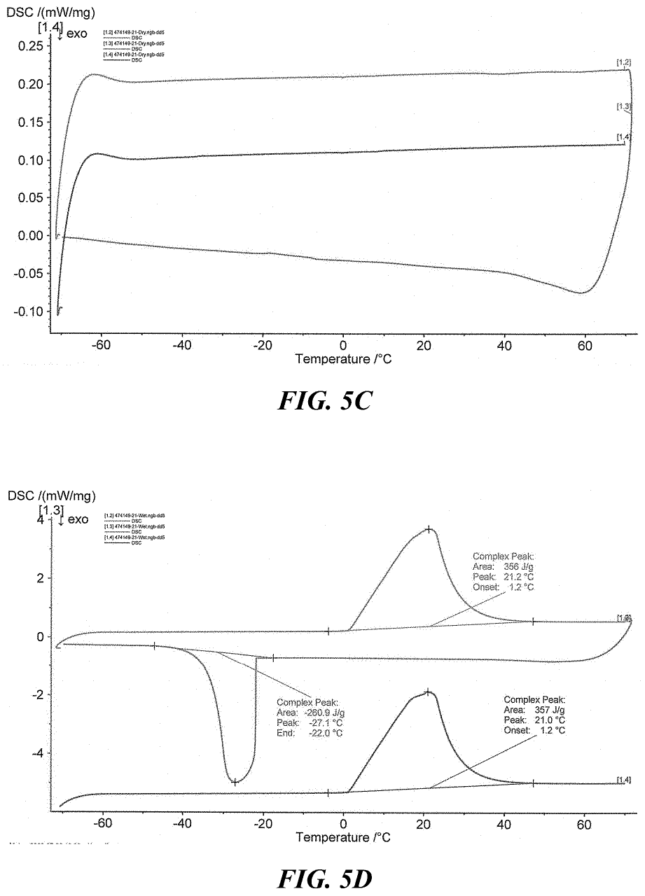

Tables 1 and 2 show the analysis conditions and summary of the data, respectively. Additional details are found in FIGS. 5A-5D.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for vaccinating a subject in need thereof comprising:
a. providing a formulation comprising a subunit vaccine molecule;
b. admixing the formulation with a mineral coated microparticle (MCM) to provide a vaccine, which MCM adsorbs the subunit vaccine molecule and has a diameter suitable for performing as an adjuvant when administered to a subject in need of vaccination; and
c. administering the vaccine to a subject in need of vaccination,
wherein a single dose of the vaccine is administered to the subject, and wherein administration of the formulation to the subject requires a plurality of administrations to be effective.

2. The method of claim 1, wherein the vaccine is injected into the subject.

3. (canceled)

4. The method of claim 1, wherein, compared with the formulation without an MCM, the vaccine has an improved bioavailability, has an improved immunogenicity, has an improved humoral response, or elicits an improved long-term memory immunity.

5. (canceled)

6. The method of claim 1, wherein the vaccine has an improved infectivity when compared with the formulation without an MCM.

7-9. (canceled)

10. The method of claim 1, wherein the subunit vaccine molecule is a protein, a peptide, or a nucleic acid.

11. (canceled)

12. The method of claim 1, wherein the MCM has a diameter less than about 100 um.

13. The method of claim 1, wherein the MCM has a core comprising calcium phosphate.

14. The method of claim 1, wherein the subunit vaccine molecule adsorbs upon and/or within a surface of the MCM.

15-21. (canceled)

22. A method for stabilizing a biological macromolecule, comprising:
a. creating a mixture comprising biological macromolecules and a mineral coated microparticles (MCM), wherein the biological macromolecule adsorbs to the MCM;
b. optionally removing biological macromolecules that are not adsorbed to the MCM from the mixture; and
c. lyophilizing the mixture to create a stabilized formulation.

23. The method of claim 22, wherein the stabilized formulation further comprises a pharmaceutically acceptable excipient material.

24. (canceled)

25. The method of claim 22, further comprising,
d. reconstituting the stabilized formulation.

26-28. (canceled)

29. The method of claim 22, wherein the biological macromolecule is a protein, a peptide, or a nucleic acid.

30-32. (canceled)

33. The method of claim 22, wherein the mixture comprises modified simulated body fluid (mSBF) comprising at least about 5 mM calcium ions and at least about 2 mM phosphate ions.

34. The method of claim 22, wherein the mixture has a pH of at least about 6.8.

35. (canceled)

36. (canceled)

37. A vaccine composition, comprising:
a. subunit vaccine molecules; and
b. mineral coated microparticles (MCM), which bind with the subunit vaccine molecules and have a diameter suitable for performing as an adjuvant when administered to a subject in need of vaccination.

38. The vaccine composition of claim 37, further comprising an adjuvant.

39-47. (canceled)

48. The vaccine composition of claim 38, wherein the adjuvant is selected from the group consisting of an aluminum, an emulsion and a salt.

49. A stabilized formulation produced by the method of claim 22.

50-52. (canceled)

53. The stabilized formulation of claim 49, wherein the formulation remains at least 90% active after six months at room temperature.

54-59. (canceled)

60. The vaccine composition of claim 37, having a longer half-life when compared with the formulation without the MCM.

61-64. (canceled)
